(19) United States
(12) Patent Application Publication
(10) Pub. No.: US 2024/0366535 A1
(43) Pub. Date: Nov. 7, 2024

(54) FORMULATION WITH IMPROVED BIOAVAILABILITY

(71) Applicant: MERRY LIFE BIOMEDICAL COMPANY, LTD., Tainan City (TW)

(72) Inventors: Hui-Chen WANG, Tainan City (TW); Chia-Yu HSU, Tainan City (TW); Ling-Ying LIAW, Tainan City (TW)

(21) Appl. No.: 18/654,677

(22) Filed: May 3, 2024

Related U.S. Application Data
(60) Provisional application No. 63/500,094, filed on May 4, 2023.

(51) Int. Cl. A61K 31/165; A61K 9/16

(52) U.S. Cl. CPC A61K 31/165; A61K 9/1617; A61K 9/1623; A61K 9/1635; A61K 9/1641; A61K 9/1652

(57) ABSTRACT

A formulation comprising TML-6 or a pharmaceutically acceptable salt, solvate, hydrate, or prodrug thereof in an amorphous form, and one or more excipients,

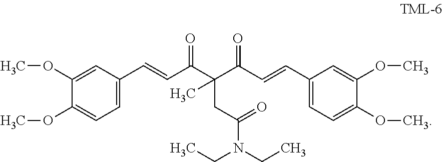 [SMILES: CCN(CC)C(=O)CC(C)(C(=O)/C=C/c1ccc(OC)c(OC)c1)C(=O)/C=C/c1ccc(OC)c(OC)c1]

### CROSS-REFERENCE TO RELATED APPLICATIONS

This application claims benefit of and priority to U.S. Provisional Patent Application No. 63/500,094, filed May 4, 2023, which is entirely incorporated herein by reference.

### FIELD OF THE INVENTION

The present disclosure invention relates to a formulation comprising a curcumin derivative. More particularly, the present disclosure relates to an oral formulation comprising a curcumin derivative, which has an improved solubility, bioavailability and/or PK profile.

### BACKGROUND OF THE INVENTION

Curcumin is a major component of Curcumin longa, generically known as turmeric, which belongs to the ginger family of perennial plants that grows naturally in India and other parts of Asia. Curcumin is effective against a wide variety of diseases like cancer, cardiovascular disease, obesity, liver disease, inflammatory disease and aging. Although the beneficial effects of curcumin have been suggested by a series of epidemiological investigations, animal experiments, and cell line studies, the low bioavailability and rapid metabolism of curcumin have hindered the therapeutic development of curcumin for human diseases [Jager R, Lowery R P, Calvanese A V, Joy J M, Purpura M, Wilson J M: Comparative absorption of curcumin formulations. Nutr J 2014, 13:11].

### SUMMARY OF THE INVENTION

The present disclosure demonstrates that a formulation comprising a curcumin derivative with improved solubility and bioavailability and/or PK profile for treat and/or prevent a neurodegenerative disease.

The present disclosure provides a formulation comprising TML-6 or a pharmaceutically acceptable salt, solvate, hydrate, or prodrug thereof in an amorphous form, and one or more excipients,

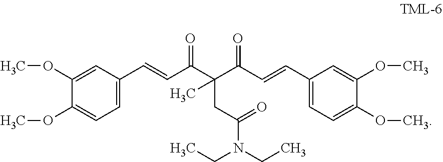 [SMILES: CCN(CC)C(=O)CC(C)(C(=O)/C=C/c1ccc(OC)c(OC)c1)C(=O)/C=C/c1ccc(OC)c(OC)c1]

In one embodiment, the formulation is characterized by a differential scanning calorimetry thermogram which does not comprise any sharp endothermic peak.

In one embodiment, the formulation is characterized by the DSC thermogram which does not comprise an endothermic peak corresponding to crystalline TML-6.

In one embodiment, the formulation is characterized by the DSC thermogram which comprises a broad endothermic peak at about 50° C. to about 90° C.

In one embodiment, the formulation is characterized by an X-ray powder diffraction (XRPD) pattern which is essentially continuous (without sharp, narrow diffraction peaks).

The formulation of claim 5, characterized by the XRPD pattern which does not comprise degree 2θ-reflections (±0.2° 2θ) at one or more of 5.5°, 11.1°, 15.7°, 16.7°, and 22.3°.

The present disclosure further provides a formulation comprising about 20% (w/w) to 90% (w/w) of TML-6, or a pharmaceutically acceptable salt, solvate, hydrate, or prodrug thereof, and about 10% (w/w) to about 80% (w/w) of one or more excipients,

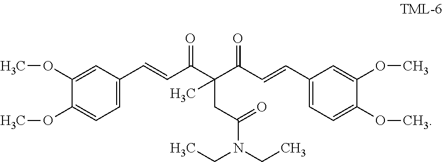 [SMILES: CCN(CC)C(=O)CC(C)(C(=O)/C=C/c1ccc(OC)c(OC)c1)C(=O)/C=C/c1ccc(OC)c(OC)c1]

In one embodiment, the TML-6, or a pharmaceutically acceptable salt, solvate, hydrate, or prodrug thereof is in an amorphous form.

In one embodiment, the formulation comprises about 70% (w/w) to 90% (w/w) of TML-6, or a pharmaceutically acceptable salt, solvate, hydrate, or prodrug thereof, and about 10% (w/w) to about 30% (w/w) of one or more excipients.

In one embodiment, the one or more excipients comprise enteric polymers.

In one embodiment, the one or more excipients comprise at least one of a polymethacrylate-based copolymer, a poly(methacrylic acid)-based copolymer, a poloxamer and a cellulose derivative excipient.

In one embodiment, the ratio of the polymethacrylate-based copolymer and/or the poly(methacrylic acid)-based copolymer to the cellulose derivative excipients ranges from about 1:5 to about 5:1.

In one embodiment, the amount of the polymethacrylate-based copolymer and/or the poly(methacrylic acid)-based copolymers range from about 10% to about 70% (w/w), and the amount of the cellulose derivative excipient ranges from about 10% to about 70% (w/w).

In one embodiment, the polymethacrylate-based copolymer and/or the poly(methacrylic acid)-based copolymer is selected from the group consisting of: Poly(methacrylic acid, methyl methacrylate) 1:1, Poly(methacrylic acid, ethyl acrylate) 1:1, Poly(methacrylic acid, methyl methacrylate) 1:2, Poly(methacrylic acid, methyl methacrylate) 1:2, Polymer of Methyl acrylate, methyl methacrylate, and methacrylic acid, Poly(butyl methacrylate-co-(2-dimethylaminoethyl) methacrylate-co-methyl methacrylate) 1:2:1, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate) with 0.7% (PEG stearyl ether) 2:1, Poly(ethyl acrylate, methyl methacrylate) with 1.5% (nonoxynol) 2:1 and Poly(ethyl acrylate, methyl methacrylate) with 1.5% (nonoxynol) 2:1 and a combination thereof.

In one embodiment, the cellulose derivative excipient is selected from the group consisting of cellulose, methyl cellulose, ethyl cellulose, carboxy methyl cellulose, hydroxypropylmethyl cellulose, cellulose ester derivative, cellulose acetate, cellulose acetate phthalate, cellulose acetate butyrate, cellulose acetate propionate, hydroxypropylmethyl cellulose phthalate, microcrystalline cellulose, hydroxyethyl cellulose and hydroxypropyl methyl cellulose.

In one embodiment, the formulation is in the form of tablets, granules, capsules or spray dried powders.

The present disclosure also provides a pharmaceutical composition comprising the formulation and one or more additional excipients.

In one embodiment, the one or more additional excipients include, but are not limited to, diluents, disintegrants, binding agents, lubricants and glidants.

The present disclosure further provides a method for preventing and/or treating a neurodegenerative disease, liver disease, cancer, cardiovascular disease, obesity, inflammatory disease or aging in a subject in need of such treatment, comprising administrating to said subject the formulation or the pharmaceutical composition.

In one embodiment, the neurodegenerative disease is amyotrophic lateral sclerosis, multiple sclerosis, Parkinson's disease, Alzheimer's disease, Huntington's disease, multiple system atrophy, dementia, motor neuron disease, Creutzfeldt-Jakob disease or prion disease.

### BRIEF DESCRIPTION OF DRAWINGS

FIG. 1 shows X-ray powder diffraction (XRPD) pattern of TML-6.

FIG. 2 shows XRPD pattern of TML-6 formulation E13.

FIG. 3 shows DSC thermograms of TML-6 and TML-6 formulations E14 to E17.

FIG. 4 shows DSC thermogram of TML-6 and TML-6 formulation E04.

FIG. 5 shows DSC thermograms of TML-6 and TML-6 formulations E10 and E12.

FIG. 6 shows plasma concentration-time profiles of TML-6 in rats (mean) following a single administration of TML-6 and TML-6 formulations E02, E10, E12 and E13 from 0 hour to 10 hours.

FIG. 7 shows plasma concentration-time profiles of TML-6 in dogs (mean) following a single administration of TML-6 and TML-6 formulations E02, E10, E12 and E13 from 0 hour to 25 hours.

### DETAILED DESCRIPTION OF THE INVENTION

The present invention can be more readily understood by reference to the following detailed description of various embodiments of the invention, the examples, and the chemical drawings and tables with their relevant descriptions. It is to be understood that unless otherwise specifically indicated by the claims, the invention is not limited to specific preparation methods, carriers or formulations, or to particular modes of formulating the compounds of the invention into products or compositions intended for topical, oral or parenteral administration, because as one of ordinary skill in the relevant arts is well aware, such things can, of course, vary. It is also to be understood that the terminology used herein is for the purpose of describing particular embodiments only and is not intended to be limiting.

In this application, terms such as “a”, “an” and “the” are not intended to refer to only a singular entity, but include the general class of which a specific example may be used for illustration. The terms “a”, “an”, and “the” are used interchangeably with the term “at least one”. The phrases “at least one of and “comprises at least one of followed by a list refers to any one of the items in the list and any combination of two or more items in the list. All numerical ranges are inclusive of their endpoints and non-integral values between the endpoints unless otherwise stated.

The term “subject” as used herein denotes any animal, preferably a mammal, and more preferably a human. Examples of subjects include humans, non-human primates, rodents, guinea pigs, rabbits, sheep, pigs, goats, cows, horses, dogs and cats.

The term “treating” or “treatment” as used herein denotes reversing, alleviating, inhibiting the progress of, or improving the disorder, disease or condition to which such term applies, or one or more symptoms of such disorder, disease or condition.

The term “polymer” refers to a molecule having a structure which includes the multiple repetition of units derived, actually or conceptually, from molecules of low relative molecular mass.

The term “solvent” refers to a homogeneous liquid material, which may be a single compound or a combination of compounds and which may or may not include water.

The term “carrier” or “excipient” as used herein refers to any substance, not itself a therapeutic agent, used as a carrier and/or diluent and/or adjuvant, or vehicle for delivery of a therapeutic agent to a subject or added to a formulation to improve its handling or storage properties or to permit or facilitate formulation of a dose unit of the composition into a discrete article such as a capsule or tablet suitable for oral administration.

As used herein, the term “pharmaceutically acceptable” is defined herein to refer to those compounds, materials, compositions and/or dosage forms, which are, within the scope of sound medical judgment, suitable for contact with the tissues a subject, e.g., a mammal or human, without excessive toxicity, irritation allergic response and other problem complications.

The compound TML-6 as described herein is a curcumin derivative or a synthetic curcumin derivative, which possesses anti-inflammatory properties involved in multiple mechanisms in AD pathogenesis, reveals a significantly better activity than curcumin in reducing brain Aβ level and anti-inflammation than curcumin. TML-6 can exist in the form of a pharmaceutically acceptable salt, solvate, hydrate, or prodrug thereof.

In some embodiments, the present disclosure provides a formulation comprising TML-6 or a pharmaceutically acceptable salt, solvate, hydrate, or prodrug thereof in an amorphous form or a crystalline form, and one or more excipients,

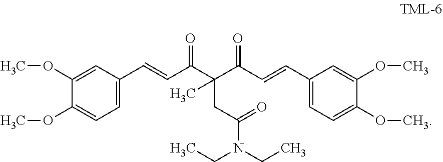 [SMILES: CCN(CC)C(=O)CC(C)(C(=O)/C=C/c1ccc(OC)c(OC)c1)C(=O)/C=C/c1ccc(OC)c(OC)c1]

In some embodiments, TML-6 is in an amorphous form. While not intending to be bound by any theory, the applicant surprisingly found that the amorphous form of TML-6 has improved solubility, dissolution and/or bioavailability. The amorphous form of TML-6 can be detected and determined by any known methods, such as X-ray powder diffraction (XRPD) and differential scanning calorimetry (DSC).

For example the formulation may be characterized by a differential scanning calorimetry thermogram which does not comprise any sharp endothermic peak, by the DSC thermogram which does not comprise an endothermic peak corresponding to crystalline TML-6 (at about 132±5° C.), or by the DSC thermogram which comprises a broad endothermic peak (e.g., at about 50° C. to about 90° C.). In some embodiments, the broad endothermic peak may be corresponding to the one or more excipients. In another aspect, the formulation may be characterized by an X-ray powder diffraction (XRPD) pattern which is essentially continuous (without sharp, narrow diffraction peaks), or by the XRPD pattern which does not comprise degree 2θ-reflections (±0.2° 2θ) at one or more of 5.5°, 11.1°, 15.7°, 16.7°, and 22.3°.

Particle size of drug, TML-6 particles size, is controlled by drying and/or milling/sieving (non-limiting examples are described below). Particle size can also be comminuted using roller compaction and milling/sieving. In the present disclosure, at least 60% of the particle size distribution of TML-6, or a pharmaceutically acceptable salt, solvate, hydrate, or prodrug thereof in the formulation is less than 150 μm. Particle size distribution can be measured using Sieve analysis, Photon Correlation Spectroscopy or laser diffraction (international standard ISO 13320-1), or electronic sensing zone, light obstruction, sedimentation or microscopy which are procedures well known by the person skilled in the art.

One or more excipients are combined with TML-6 to prepare the formulation as described herein. The formulation as described herein comprises about 20% (w/w) to 90% (w/w) of TML-6, or a pharmaceutically acceptable salt, solvate, hydrate, or prodrug thereof, and about 10% (w/w) to about 80% (w/w) of one or more excipients. In some embodiments, the formulation may comprise about 70% (w/w) to 90% (w/w) of TML-6, or a pharmaceutically acceptable salt, solvate, hydrate, or prodrug thereof, and about 10% (w/w) to about 30% (w/w) of one or more excipients.

The one or more excipients comprise one or more polymethacrylate-based copolymers, poly(methacrylic acid)-based copolymers, poloxamer and/or cellulose derivative excipients. While not intending to be bound by any theory, it is believed that these excipients can increase dissolution of TML-6 in gastric juice and/or intestinal juice, control solid form of TML-6 (preferably in amorphous form) and/or improve PK profile of TML-6 formulations. Preferably, the amount of polymethacrylate-based copolymer ranges from about 10% to about 70% (w/w) and the amount of cellulose derivative excipient from about 10% to about 70% (w/w). The polymethacrylate-based copolymer includes poly(methyl methacrylate) (PMMA) based copolymers. Certain embodiments of the PMMA based copolymers include, but are not limited to, neutral, cationic, and anionic forms of PMMA based copolymers. In some embodiments, the PMMA based copolymers may include a copolymer based on methyl methacrylate and one or more of methacrylic acid, methacrylic esters, and acrylic esters. In a further embodiment, the PMMA is poly(methacrylic acid-co-methyl methacrylate). In some further embodiments, the poly(methacrylic acid-co-methyl methacrylate includes, but is not limited to, any one of the polymer based on methacrylic acid and methyl methacrylate as described herein.

Certain embodiments of poly(methacrylic acid)-based copolymers include, but are limited to, copolymers of methacrylic acid with methyl acrylate, ethyl acrylate, tert-butyl acrylate, methyl methacrylate, and N-isopropylacrylamide. In further embodiments, the poly(methacrylic acid)-based copolymers may be methacrylic acid-methyl methacrylate copolymer (1:1) and/or methacrylic acid-ethyl acrylate copolymer (1:1).

Certain embodiments of cellulose derivative excipient include, but are not limited to, cellulose, methyl cellulose, ethyl cellulose, carboxy methyl cellulose, hydroxypropylmethyl cellulose, cellulose ester derivative, cellulose acetate, cellulose acetate phthalate, cellulose acetate butyrate, cellulose acetate propionate, hydroxypropylmethyl cellulose phthalate, microcrystalline cellulose (MCC), hydroxyethyl cellulose and hydroxypropyl methyl cellulose.

In some embodiments, the polymethacrylate-based copolymer and/or the poly(methacrylic acid)-based copolymer includes Poly(methacrylic acid, methyl methacrylate) 1:1, Poly(methacrylic acid, ethyl acrylate) 1:1, Poly(methacrylic acid, methyl methacrylate) 1:2, Poly(methacrylic acid, methyl methacrylate) 1:2, Polymer of Methyl acrylate, methyl methacrylate, and methacrylic acid, Poly(butyl methacrylate-co-(2-dimethylaminoethyl) methacrylate-co-methyl methacrylate) 1:2:1, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate) with 0.7% (PEG stearyl ether) 2:1, Poly(ethyl acrylate, methyl methacrylate) with 1.5% (nonoxynol) 2:1 and Poly(ethyl acrylate, methyl methacrylate) with 1.5% (nonoxynol) 2:1 and a combination thereof.

In some embodiments, the ratio of the polymethacrylate-based copolymers and/or the poly(methacrylic acid)-based copolymers to the cellulose derivative excipients ranges from about 1:5 to about 5:1. For example, the ratio of the poly(methacrylic acid)-based copolymer to the cellulose derivative excipient ranges from about 1:5 to about 5:1, such as 5:25, 6:24, 7:23, 8:22, 9:21, 10:20, 11:19, 12:18, 13:17, 14:16, 15:15, 16:14, 17:13, 18:12, 19:11, 20:10, 21:9, 22:8, 23:7, 24:6 or 25:5.

In some embodiments, the one or more excipients comprises one or more polymeric materials, such as polysorbate and/or poloxamer. Certain embodiments of polysorbate include, but are not limited to, tween 20, tween 40, tween 60, tween 80 and tween 85. Certain embodiments of poloxamer include, but are not limited to, poloxamer 124, poloxamer 188, poloxamer 237, poloxamer 338 and poloxamer 407. These polymeric materials can be used alone or with one or more polymethacrylate-based copolymers and/or cellulose derivative excipients.

The formulation as described herein can be prepared by ball milling, wet granulation, direct compression and dry granulation, hot melt extrusion or spray drying. In some embodiment, the formulation may be in form of granules, spray dried powders, capsules or tablets.

The formulation as described herein can be optionally combined with one or more additional excipients to form a pharmaceutical composition. In one embodiment, the pharmaceutical composition can be a tablet or capsule. Certain embodiments of the one or more additional excipients include, but are not limited to, diluents, disintegrants, binders, lubricants and glidants.

Lubricants are typically added to prevent the tableting materials from sticking to punches, minimize friction during tablet compression, and allow for removal of the compressed tablet from the die. Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose.

Binders are agents, which impart cohesive qualities to the powdered material. Commonly used binders include starch, and sugars, such as sucrose, glucose, dextrose and lactose. Examples of pharmaceutically acceptable binders include, but are not limited to, starches; celluloses and derivatives thereof, e.g., microcrystalline cellulose, hydroxypropyl cellulose hydroxylethyl cellulose and hydroxylpropylmethyl cellulose; sucrose; dextrose; corn syrup; polysaccharides; and gelatin.

Diluents which are added to increase the bulk weight of the blend resulting in a practical size for compression. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc.

Disintegrants are often included to ensure that the tablet has an acceptable rate of disintegration. Typical disintegrants include starch derivatives and salts of carboxymethylcellulose. Examples of pharmaceutically acceptable disintegrants include, but are not limited to, starches; clays; celluloses; alginates; gums; cross-linked polymers, e.g., cross-linked polyvinyl pyrrolidone (VPV), cross-linked calcium carboxymethylcellulose and cross-linked sodium carboxymethylcellulose; soy polysaccharides; and guar gum.

Examples of glidants include, but are not limited to, magnesium stearate, calcium stearate, zinc stearate and sodium stearyl fumarate.

Other examples of useful excipients are described in the Handbook of pharmaceutical excipients, 3rd edition, Edited by A. H. Kibbe, Published by: American Pharmaceutical Association, Washington DC, ISBN: 0-917330-96-X, or Handbook of Pharmaceutical Excipients (4th edition), Edited by Raymond C Rowe—Publisher: Science and Practice.

The present disclosure also provides a method for preventing and/or treating a neurodegenerative disease, liver disease, cancer, cardiovascular disease, obesity, inflammatory disease or aging in a subject in need of such treatment, comprising administrating to said subject the formulation or pharmaceutical composition as described herein. Applications of TML-6 for treatment of these diseases are those as discussed in Min Hao, Yue Chu, Jingxuan Lei, Zhouhui Yao, Pingping Wang, Ziyan Chen, Kuilong Wang, Xianan Sang, Xin Han, Lu Wang, Gang Cao. Pharmacological Mechanisms and Clinical Applications of Curcumin: Update. Aging and disease. 2023, 14(3): 716-749 https://doi.org/10.14336/AD.2022.1101, and Su I J, Chang H Y, Wang H C, Tsai K J. A Curcumin Analog Exhibits Multiple Biologic Effects on the Pathogenesis of Alzheimer's Disease and Improves Behavior, Inflammation, and β-Amyloid Accumulation in a Mouse Model. Int J Mol Sci. 2020 Jul. 30; 21(15):5459. doi: 10.3390/ijms21155459. PMID: 32751716; PMCID: PMC7432838. Examples of the neurodegenerative disease include, but are not limited to, amyotrophic lateral sclerosis, multiple sclerosis, Parkinson's disease, Alzheimer's disease, Huntington's disease, multiple system atrophy, dementia, motor neuron disease, Creutzfeldt-Jakob disease and prion diseases.

The formulation and pharmaceutical composition as described herein have improved solubility and bioavailability and thus confers advantageous efficacy in preventing and/or treating a neurodegenerative disease.

The following examples/studies are provided to aid those skilled in the art in practicing the present invention.

### Preparation of Formulation of the Present Disclosure: Wet Granulation

For formulations Examples 1 to 3 (E01 to E03), the components list in Table 1 were sieved and mixed to form granules, and then dried and shaped to form the formulation products. In Table 1, TPGS is the abbreviation for D-α-Tocopherol polyethylene glycol-1000 succinate, and SDS is the abbreviation for sodium dodecyl sulfate (also referred to as SLS).

TABLE 1
Components of formulations (E01 to E03).

| Components (wt %) | E01 | E02 | E03 |
|---|---|---|---|
| TML-6 | 50 | 50 | 50 |
| Lactose 200 | 17 | 20 | 7 |
| TPGS | 25 | 25 | |
| Tween 80 | | | 10 |
| Kollidone VA64 | | | |
| PVP K30 | 3 | | 3 |
| SDS | | | 25 |
| Crospovidone | 5 | 5 | 5 |

### Preparation of Formulation of the Present Disclosure: Solvent Evaporation Solid Dispersion

For formulations of Examples 4 to 12 (E04 to E12), the components list in Table 2 were dispersed in the solvent (purified water, 9500 ethanol and/or acetone), and the resulting dispersions was dried in an oven. After cooling, the dry dispersion was grind and sieved.

TABLE 2
Components of formulations (E04 to E12).

| Components (wt %) | E04 | E05 | E06 | E07 | E08 | E09 | E10 | E11 | E12 |
|---|---|---|---|---|---|---|---|---|---|
| TML-6 | 47.50 | 33.33 | 22.22 | 33.33 | 18.18 | 23.70 | 23.46 | 31.00 | 29.33 |
| Poly(butyl methacrylate-co-(2- | 47.50 | | | | | | | | |
| dimethylaminoethyl) | | | | | | | | | |
| methacrylate-co-methyl | | | | | | | | | |
| methacrylate) 1:2:1 | | | | | | | | | |
| HPMC E5 LV | | | | | 36.36 | 47.40 | 46.92 | | |
| Poloxamer 188 | | | | | 18.18 | 23.70 | 23.46 | 62.00 | 58.65 |
| Tween 80 | | | | | | | | 1.00 | 1.00 |
| Kollidone VA64 | | | | 33.33 | | | | | |
| PVP K30 | | 33.33 | 44.44 | | | | | | |
| SDS | | | | | | | | | 5.00 |
| MCC102 | | | 33.34 | 33.34 | 27.38 | | | | |
| MCC101 | | 25.00 | | | | | | | |
| Aerosil 200 | | | | | | | 1.00 | 1.00 | 1.00 |
| Crospovidone | 5.00 | | | | | 5.20 | 5.16 | 5.00 | 5.02 |

### Preparation of Formulation of the Present Disclosure: Spray Drying

The components of the formulation of Example 13 (E13) is as shown in Table 1

TABLE 3
Components of formulation (E13).

| | |
|---|---|
| API | TML-6, 10.5 g |
| Polymer | methacrylic acid-ethyl acrylate copolymer |
| | (1:1) 19.5 g |
| Solvent | Acetone /EtOH = 95/5 v/v % 1500 mL |
| Ratio: API/Polymer | 35/65 w/w %, solid cone.: 2% in solvent |

The above components were mixed and the resulting mixture was sprayed using spray dryer and then collected the spray dried powder.

The formulations of Examples 14 to 17 were prepared with the same spray drying method, and the components thereof are as shown in Table 4.

TABLE 4
Components of formulations (E13 to E17).

| Components (wt %) | E13 | E14 | E15 | E16 | E17 |
|---|---|---|---|---|---|
| TML-6 | 35 | 70 | 80 | 70 | 70 |
| Poly(methacrylic | 65 | 30 | 20 | 10 | |
| acid-co-ethyl acrylate) 1:1 | | | | | |
| HPMC | | | | 20 | 30 |

### X-Ray Powder Diffraction (XRPD) Studies

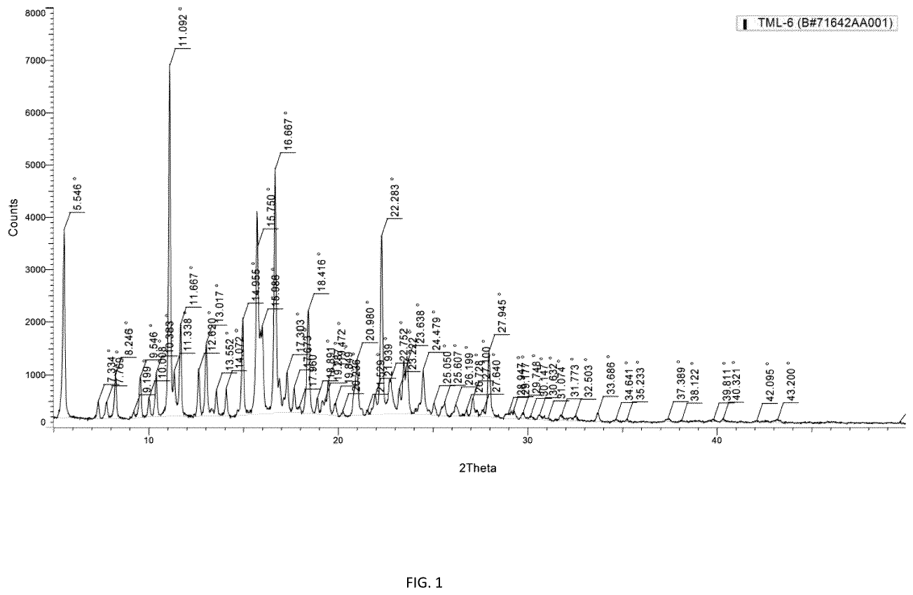

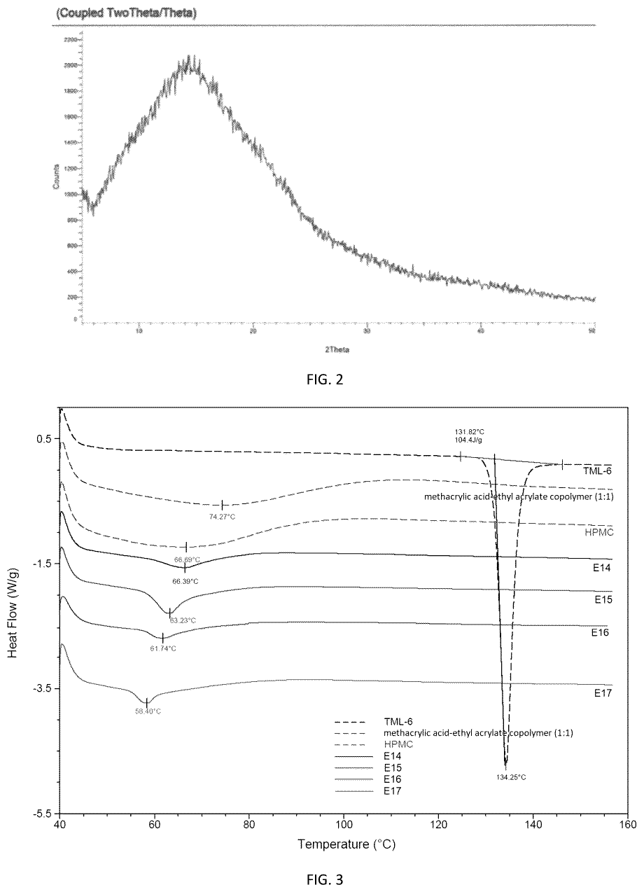

TML-6 (unformulated) and TML-6 formulation E13 were analyzed on the zero background sample holder and present it for X-ray analysis. The XRPD pattern of TML-6 (FIG. 1) showed degree 2θ-reflections (±0.2° 2θ) at 5.5°, 11.1°, 15.7°, 16.7°, 22.3°, etc., which indicated the crystalline state.

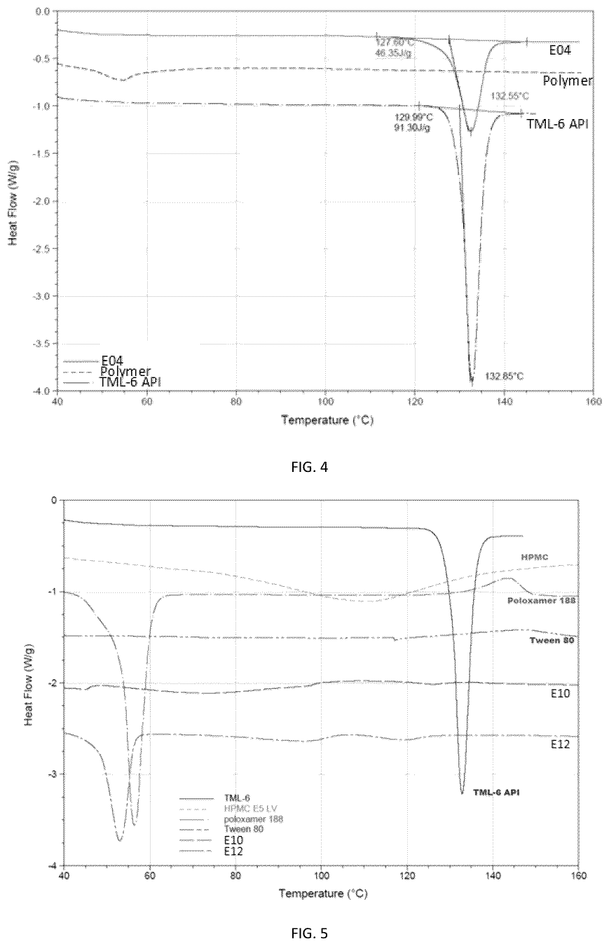

The XRPD pattern of TML-6 formulation E13 (FIG. 4) was essentially continuous without sharp, narrow diffraction peaks, which indicated an amorphous form.

### Differential Scanning Calorimetry (DSC) Studies

The sample (1-2 mg) was accurately weighed into aluminum pan and sealed, and are heated from 40 to 160° C. with a heating ramp of 10° C./min for DSC analysis.

The DSC thermogram of TML-6 (FIG. 3) was characterized by one sharp endothermic peak at 134.25° C., corresponding to its melting point at the crystalline state.

The DSC thermograms of TML-6 formulation E14 to E17 (FIG. 3) showed a broad peak at about 50° C. to about 90° C., corresponding to the melting point of the excipients (may shift), and the characteristic peak of the crystalline TML-6 is not displayed.

The DSC melting points of TML-6 formulations E10 and E12 (FIG. 5) showed no significant exothermic or endothermic peak, which also indicated an amorphous form.

The DSC thermogram of TML-6 formulation E04 (FIG. 4) showed an endothermic peak at 132.55° C., close to that of TML-6 (about 132.85° C.), which indicated the crystalline state.

### Dissolution Test in 0.1 HCl and pH 6.8 Phosphate Buffer, 30 Min

The dissolution of TML-6 (API alone) and TML-6 formulations (E01 to E12) were measured in mediums simulating gastric juice for 30 mins, and the results are as shown in Table 5 and 6. The dissolution of TML-6 (API alone) and TML-6 formulations (E13 to E17) were measured in mediums simulating intestinal juice for 30 mins, and the results are as shown in Table 6.

TABLE 5
Dissolution of TML-6 formulations (E01 to E12)

| | | | Dissolution | |
|---|---|---|---|---|
| | Formulation Carrier/polymer Ratio | Solid form | within 30 min | Medium |
| API alone | TML-6 | Crystalline | 0.1% | 0.1N HCl |
| E01 | TML-6:TPGS (2:1) + PVP K30 | Crystalline | 1.1% | 0.1N HCl |
| E02 | TML-6:TPGS (2:1) | Crystalline | 1.1% | 0.1N HCl |
| E03 | TML-6:SLS:Tween 80 (1:0.5:0.2) + PVP | Crystalline | 1.1% | 0.1N HCl |
| | K30 | | | |
| E04 | TML-6:Poly(butyl methacrylate-co-(2- | Crystalline | 6.1% | 0.1N HCl |
| | dimethylaminoethyl) methacrylate-co-methyl | | | |
| | methacrylate) 1:2:1 (1:1) | | | |
| E05 | TML-6:PVP K30 (1:1) | Crystalline | 3.7% | 0.1N HCl + |
| | | | | 0.2% SLS |
| E06 | TML-6:PVP K30 (1:2) | Crystalline | 2.6 | 0.1N HCl + |
| | | | | 0.2% SLS |
| E07 | TML-6:Kollidone VA64 (1:1) | Crystalline | 3.9% | 0.1N HCl + |
| | | | | 0.2% SLS |
| E08 | TML-6:HPMC:P188 (1:2:1) | Amorphous | 60.1% | 0.1N HCl + |
| | | | | 0.2% SLS |
| E09 | TML-6:HPMC:P188 (1:2:1) | Amorphous | 68.2% | 0.1N HCl + |
| | | | | 0.5% SLS |
| E10 | TML-6:HPMC:P188 (1:2:1) | Amorphous | 88.5% | 0.1N HCl + |
| | | | | 0.2% SLS |
| E11 | TML-6:Poloxamer 188 (1:2) | Amorphous | 85.0% | 0.1N HCl + |
| | | | | 0.2% SLS |
| E12 | TML-6:HPMC:P188 (1:2:1) | Amorphous | 87.7% | 0.1N HCl + |
| | | | | 0.2% SLS |

TABLE 6
Dissolution of TML-6 formulations (SD0 to SD3 and SD5)

| | Formulation Ratio | | | |
|---|---|---|---|---|
| | TML-6:methacrylic | | Dissolution | |
| | acid-ethyl acrylate | | within | |
| | copolymer (1:1):HPMC | Solid form | 30 mins | Medium |
| API | 100:0:0 | Crystalline | 0.06% | pH 6.8 |
| alone | | | | Phosphate |
| | | | | Buffer |
| E13 | 35:65:0 | Amorphous | 98.7% | pH 6.8 |
| E14 | 70:30:0 | Amorphous | 97.7% | Phosphate |
| E15 | 80:20:0 | Amorphous | 93.6% | Buffer + |
| E16 | 70:10:20 | Amorphous | 57.5% | 1% SLS |
| E17 | 70:0:30 | Amorphous | 39.8% | |

Referring to Tables 5 and 6 above, addition a polymethacrylate-based copolymer, a poly(methacrylic acid)-based copolymer, a poloxamer and/or a cellulose derivative excipient can effectively improve dissolution of TML-6. Besides, TML-6 shows an improved dissolution in amorphous form than in crystalline form.

### Pharmacokinetics (PK) Studies in Rats

The objective of this study is to obtain the pharmacokinetic profiles of TML-6 API (unformulated, crystalline), TML-6 formulation E02 (crystalline) and TML-6 formulations E10, E12 and E13 (amorphous) though oral (PO) doses when administrated to 6-8 week old male Sprague-Dawley rats (BioLASCO Taiwan Co. Ltd). A study design of five doses on rats. Animals received a single dose of TML-6 API, TML-6 formulations E02, E10, E12 and E13 at 25 mg/kg via oral gavage.

Dosing Solution Preparation: The TML-6 API, E02, E10, E12 and E13 oral formulations were prepared by suspending these TML6 formulated powder in the aqueous vehicle composed of 0.5% methylcellulose (MC) to the concentration of 2.5 mg/mL TML-6, respectively.

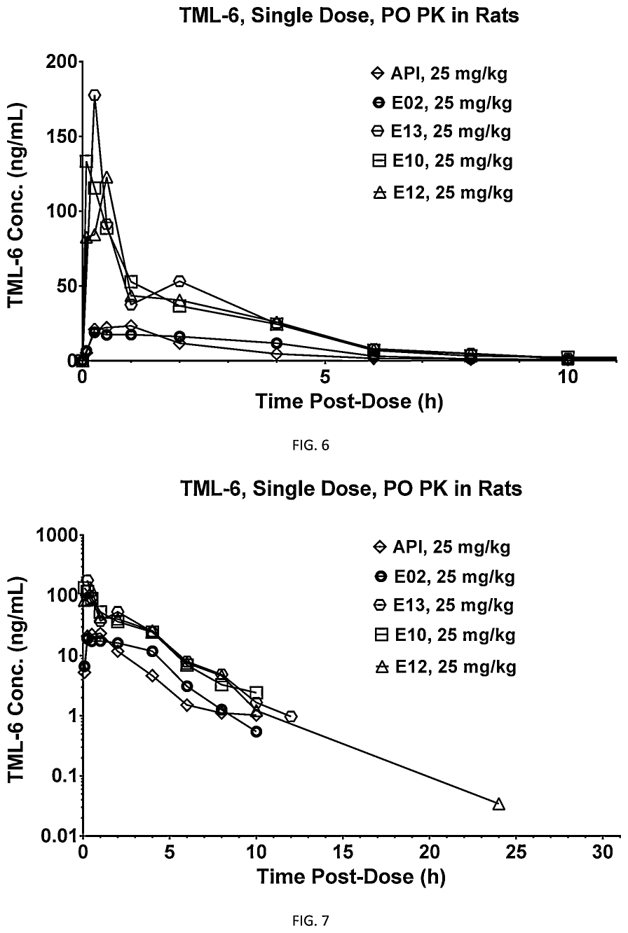

Plasma Sample Preparation and Bioanalysis Serial blood samples were collected from all animals through the tail veins. Approximately 0.2 mL blood samples were collected at each following time points: predose and 0.083, 0.25, 0.5, 1, 2, 4, 6, 8, 10 and 24 hours post-dose. Separation of plasma was prepared by centrifugation (1,000×g for 15 minutes at 4° C.) after blood sampling. Concentrations of TML-6 in rat plasma were determined by liquid chromatography-tandem mass spectrometry (LC-MS/MS), as shown in FIGS. 6 and 7. The lower limit of quantification (LLOQ) was 0.1 ng/mL. The plasma concentration-time data above the LLOQ at each dose level were used in the calculation of PK parameters of TML-6 (Table 7).

RESULTS: PK studies of TML-6 API and TML-6 formulations E02, E10, E12 and E13 were conducted following orally administered 25 mg/kg in 0.5% MC suspension to rats. The Cₘₐₓ and AUC_{(0-∞)} of ML-6 API and TML-6 formulations E02, E10, E12 and E13 were summarized in Table 7. There were no significant differences in Cₘₐₓ and AUC_{(0-∞)} among unformulated TML-6 (TML-6 API) and TML-6 formulation E02. TML-6 formulations E10, E12 and E13 can help to improved Cₘₐₓ and AUC_{(0-∞)} of TML-6. The results also showed that the formulations with improved dissolution of TML-6 also have improved Cₘₐₓ and AUC_{(0-∞)}.

TABLE 7
Summary of Cₘₐₓ and AUC_{(0-∞)} of TML-6 in
Rats Following Administration of Oral Doses.

| | | | Dose | Cₘₐₓ | AUC_{(0-∞)} |
|---|---|---|---|---|---|
| | Route | Solid form | (mg/kg) | (ng/mL) | (ng*hr/mL) |
| API alone | PO | crystalline | 25.0 | 25.3 | 73.6 |
| E02 | PO | crystalline | 25.0 | 19.8 | 82.9 |
| E10 | PO | Amorphous | 25.0 | 142 | 256 |
| E12 | PO | Amorphous | 25.0 | 144 | 250 |
| E13 | PO | Amorphous | 25.0 | 178 | 267 |

The compositions and methods of the disclosure each have several innovative aspects, no single one of which is solely responsible or required for the desirable attributes disclosed herein. The various features and processes described above may be used independently of one another, or may be combined in various ways. All possible combinations and subcombinations are intended to fall within the scope of this disclosure. Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. No single feature or group of features is necessary or indispensable to each and every embodiment.

1. A formulation comprising TML-6 or a pharmaceutically acceptable salt, solvate, hydrate, or prodrug thereof in an amorphous form, and one or more excipients,

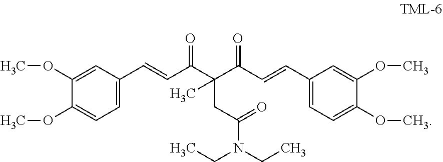 [SMILES: CCN(CC)C(=O)CC(C)(C(=O)/C=C/c1ccc(OC)c(OC)c1)C(=O)/C=C/c1ccc(OC)c(OC)c1]

2. The formulation of claim 1, characterized by a differential scanning calorimetry thermogram which does not comprise any sharp endothermic peak.

3. The formulation of claim 1, characterized by the DSC thermogram which does not comprise an endothermic peak corresponding to crystalline TML-6.

4. The formulation of claim 1, characterized by the DSC thermogram which comprises a broad endothermic peak at about 50° C. to about 90° C.

5. The formulation of claim 1, characterized by an X-ray powder diffraction (XRPD) pattern which is essentially continuous (without sharp, narrow diffraction peaks).

6. The formulation of claim 5, characterized by the XRPD pattern which does not comprise degree 2θ-reflections (±0.2° 2θ) at one or more of 5.5°, 11.1°, 15.7°, 16.7°, and 22.3°.

7. A formulation comprising about 20% (w/w) to 90% (w/w) of TML-6, or a pharmaceutically acceptable salt, solvate, hydrate, or prodrug thereof, and about 10% (w/w) to about 80% (w/w) of one or more excipients,

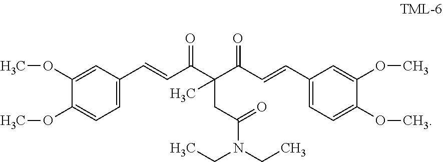 [SMILES: CCN(CC)C(=O)CC(C)(C(=O)/C=C/c1ccc(OC)c(OC)c1)C(=O)/C=C/c1ccc(OC)c(OC)c1]

8. The formulation of claim 7, wherein the TML-6, or a pharmaceutically acceptable salt, solvate, hydrate, or prodrug thereof is in an amorphous form.

9. The formulation of claim 7, which comprises about 70% (w/w) to 90% (w/w) of TML-6, or a pharmaceutically acceptable salt, solvate, hydrate, or prodrug thereof, and about 10% (w/w) to about 30% (w/w) of one or more excipients.

10. The formulation of claim 7, wherein the one or more excipients comprise enteric polymers.

11. The formulation of claim 7, wherein the one or more excipients comprise at least one of a polymethacrylate-based copolymer, a poly(methacrylic acid)-based copolymer, a poloxamer and a cellulose derivative excipient.

12. The formulation of claim 11, wherein the ratio of the polymethacrylate-based copolymer and/or the poly(methacrylic acid)-based copolymer to the cellulose derivative excipients ranges from about 1:5 to about 5:1.

13. The formulation of claim 11, wherein the amount of the polymethacrylate-based copolymer and/or the poly(methacrylic acid)-based copolymers range from about 10% to about 70% (w/w), and the amount of the cellulose derivative excipient ranges from about 10% to about 70% (w/w).

14. The formulation of claim 11, wherein the polymethacrylate-based copolymer and/or the poly(methacrylic acid)-based copolymer is selected from the group consisting of: Poly(methacrylic acid, methyl methacrylate) 1:1, Poly(methacrylic acid, ethyl acrylate) 1:1, Poly(methacrylic acid, methyl methacrylate) 1:2, Poly(methacrylic acid, methyl methacrylate) 1:2, Polymer of Methyl acrylate, methyl methacrylate, and methacrylic acid, Poly(butyl methacrylate-co-(2-dimethylaminoethyl) methacrylate-co-methyl methacrylate) 1:2:1, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.2, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate, 2-trimethylammonioethyl methacrylate chloride or 2-(methacryloyloxy)-N,N,N-trimethylethanaminium chloride) 1:2:0.1, Poly(ethyl acrylate, methyl methacrylate) with 0.7% (PEG stearyl ether) 2:1, Poly(ethyl acrylate, methyl methacrylate) with 1.5% (nonoxynol) 2:1 and Poly(ethyl acrylate, methyl methacrylate) with 1.5% (nonoxynol) 2:1 and a combination thereof.

15. The formulation of claim 7, wherein the cellulose derivative excipient is selected from the group consisting of cellulose, methyl cellulose, ethyl cellulose, carboxy methyl cellulose, hydroxypropylmethyl cellulose, cellulose ester derivative, cellulose acetate, cellulose acetate phthalate, cellulose acetate butyrate, cellulose acetate propionate, hydroxypropylmethyl cellulose phthalate, microcrystalline cellulose, hydroxyethyl cellulose and hydroxypropyl methyl cellulose.

16. The formulation of claim 7, wherein the formulation is in the form of tablets, granules, capsules or spray dried powders.

17. A pharmaceutical composition comprising the formulation of claim 7 and one or more additional excipients.

18. The pharmaceutical composition of claim 17, wherein the one or more additional excipients include, but are not limited to, diluents, disintegrants, binding agents, lubricants and glidants.

19. A method for preventing and/or treating a neurodegenerative disease, liver disease, cancer, cardiovascular disease, obesity, inflammatory disease or aging in a subject in need of such treatment, comprising administrating to said subject the pharmaceutical composition of claim 17.

20. The method of claim 19, wherein the neurodegenerative disease is amyotrophic lateral sclerosis, multiple sclerosis, Parkinson's disease, Alzheimer's disease, Huntington's disease, multiple system atrophy, dementia, motor neuron disease, Creutzfeldt-Jakob disease or prion disease.
